(19) United States
(12) Patent Application Publication
(10) Pub. No.: US 2008/0312514 A1
(43) Pub. Date: Dec. 18, 2008

(54) Serum Patterns Predictive of Breast Cancer

(76) Inventor: Brian C. Mansfield, Catonsville, MD (US)

Correspondence Address: COOLEY GODWARD KRONISH LLP;ATTN: Patent Group, Suite 1100, 777 - 6th Street, NW, WASHINGTON, DC, 20001, US

(21) Appl. No.: 11/914,091

(22) PCT Filed: May 12, 2006
(86) PCT No.: PCT/US2006/018486

§ 371 Date: Jul. 9, 2008

Related U.S. Application Data
(60) Provisional application No. 60/679,989, filed on May 12, 2005.

(51) Int. Cl. A61B 5/00

(52) U.S. Cl. 600/300

(57) ABSTRACT

Models for classifying a biological sample are developed from samples taken from a mammalian subject into one of at least two possible biological states related to breast cancer. Samples may be processed by mass spectral and other high-throughput analytical techniques.

### PRIORITY CLAIM

The present application claims priority to U.S. Provisional Application Ser. No. 60/679,989, filed May 12, 2005, and hereby incorporates by reference the entire disclosure thereof.

### FIELD OF THE INVENTION

The present invention relates to diagnostic methods that are predictive of malignancies, particularly breast cancer.

### BACKGROUND

Methods of analyzing biological samples through the identification of specific biomarkers are generally known. Because relative changes in markers (or features) of complex biological samples are typically subtle and difficult to perceive by visual examination, pattern recognition technologies are of increasing interest in the diagnostic field. See U.S. Pat. No. 6,925,389 and Published Application 2002/0046198. When combined with powerful data-mining algorithms, coordinated changes in multiple molecular species, e.g., as found in serum, can be correlated with various diseases such as malignancy.

In an exemplary analysis, a high-throughput bioassay, such as mass spectroscopy, NMR or electrophoresis may be performed on the biological sample to separate and quantify at least some of its constituent molecular components (e.g., proteins, protein fragments, DNA, RNA, etc.). Based on the output of the bioassay, such as a mass spectrum, various diagnostics may be run. For example, a diagnostic model of a particular disease state may be applied to the mass spectrum to identify the sample from which the spectrum was derived as being taken from a subject that has, is suspected of having or is at risk of having the disease state.

Some of the known methods of analyzing biological samples accomplish simultaneously (or at the same or different sites) the acquisition of patient-specific data (i.e., the performance of a high-throughput bioassay) and the analysis of the data (i.e., the application of the diagnostic model). See, for example, U.S. patent application Ser. No. 11/008,784.

### SUMMARY OF THE INVENTION

Models for classifying a biological sample are developed from samples taken from a mammalian subject into one of at least two possible biological states related to breast cancer. Samples may be processed by mass spectral and other high-throughput analytical techniques. A model includes at least one classifying hypervolume associated with one of the at least two biological states related to breast cancer and disposed within a vector space having n dimensions, each dimension corresponding to a different mass-to-charge value, where n is at least three and at least a first of the dimensions corresponds to a mass-to-charge value in a range of m/z values selected from the m/z ranges consisting of between 200 to 300, 300 to 400, 400 to 500, 500 to 600, 600 to 700, and 700 to 900.

### BRIEF DESCRIPTION OF DRAWINGS

FIG. 1 shows a distribution of features across many models.

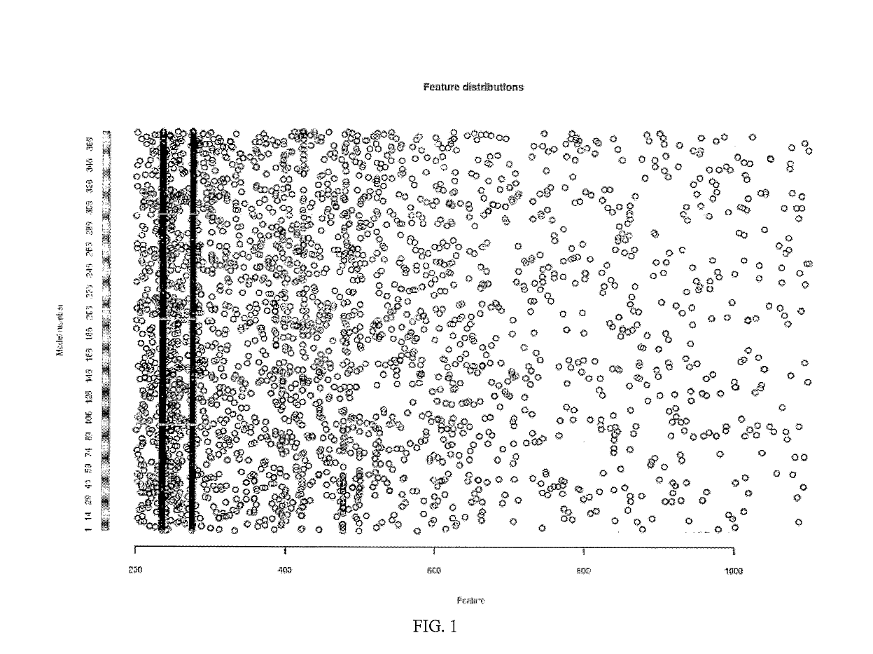

### DETAILED DESCRIPTION

The multi factor nature and progression of cancer and other diseases suggests that single biomarkers may not be accurate predictors of disease, disease progression and responsiveness to treatment. However, the pattern formed by a combination of several biomarkers could result in both early detection and more accurate diagnosis. To identify such “fingerprints” it is advantageous to use high throughput serum profiling combined with powerful bioinformatics tools for data processing, analysis and pattern recognition.

Using computational technologies, a diagnostic model can be built to determine if a biological sample exhibits or is predictive or suggestive of a particular biological state. Such states may be associated with one or more diseases or physiological status. To produce such a model, a number of samples having a known biological state can be analyzed and compared with samples known to have been taken from patients who do not have that biological state. These data are then input into a modeling program to find discriminatory patterns that are specific to a particular biological state. Such patterns are based upon various combinations of features or markers found in the data derived from the samples.

An example of diagnostic modeling and pattern recognition technology that may be used to determine whether a sample has a particular biological state is the Knowledge Discovery Engine (“KDE”), which is disclosed in U.S. patent application Ser. No. 09/883,196, now U.S. Application Publication No. 2002/0046198A1, entitled “Heuristic Methods of Classification,” filed Jun. 19, 2001 (“Heuristic Methods”), and U.S. patent application Ser. No. 09/906,661, now U.S. Application Publication No. 2003/0004402, entitled “A Process for Discriminating Between Biological States Based on Hidden Patterns L from Biological Data,” filed Jul. 18, 2001 (“Hidden Patterns”). Software implementing the KDE is available from Correlogic Systems, Inc. under the name Proteome Quest. Related technologies and associated equipment platforms include the Biomarker Amplification Filter Technology of Predictive Diagnostics, Inc. as described in U.S. Pat. No. 6,980,674 and the ProteinChip System of Ciphergen Biosystems, Inc.

After being developed, a diagnostic model may be used to determine if a new biological sample whose state is unknown exhibits a particular biological state. Data characterizing the biological sample (e.g. from a bioassay such as a mass spectrum) can be compared to the model. When the pattern recognition technology is the KDE described above, an assessment can be made of whether data that is abstracted from or that characterizes the sample falls within one of the diagnostic clusters that make up the models produced by that technology.

The entire disclosure of each document identified herein is hereby incorporated by reference.

### EXAMPLE 1

The study described in this Example 1 used serum collected as part of the Clinical Breast Care Project at the Walter Reed Army Medical Center.

Key components of the study are:

Standardized pre-operative serum collection protocols applied to both retrospective and prospective samples.

A prospective, multi-site collection to accrue 1,000 independent serum samples from women with normal/benign breast condition and 1,000 independent serum samples from women with breast cancer.

A sample set encompassing the geographic and ethnic diversity of the broad DS population.

Detailed post-operative pathology reports of patient age, menopausal status and diagnosis, tumor stage, size, grade, receptor status, comedo, nuclear grade, necrosis, and distribution allowing groupings by multiple criteria. Initial grouping/modeling is by:
normal breast condition
benign, non-neoplastic breast condition
benign, neoplastic breast condition
in situ DCIS, LCIS
invasive carcinoma

High throughput, high resolution mass spectrometry.

The ProteomeQuest® Pattern Recognition Software Package.

The current status of the serum collection is shown in Tables 1 and 2.

### Methods

691 serum samples were analyzed from women with a breast abnormality (clinical or radiologic) undergoing breast biopsy. Sera samples were from: 32 no breast disease; 204 benign non-neoplastic conditions; 111 benign neoplastic conditions; 24 atypical ductal hyperplasia only; 234 invasive cancer; 86 in situ carcinoma, (61 ductal carcinoma in situ (“DCIS”) and 25 lobular carcinoma in situ (“LCIS”)).

Sera were collected prior to biopsy, and processed promptly according to a standard protocol. Pathology of tissue biopsy was used to classify samples. Sera were analyzed on an ABI QSTAR time-of-flight mass spectrometer equipped with an Advion Nanomate® System. Spectra obtained were used to build models using the Correlogic Systems Inc. ProteomeQuest® software which combines lead cluster mapping with a genetic algorithm to identify patterns predictive of disease status.

We held an independent set of spectra files out from model development as a blinded validation set to emulate a clinical setting.

### Results

A number of models were created which demonstrated sensitivities and specificities in the range of 80-90% on the blinded validation set. We identified three regions in the spectra that together contain at least 8 m/z features, which are very powerful in discriminating between invasive cancer and non-malignant conditions.

Singly, the features are not very informative, but combined in a multi-dimensional model to reflect coordinated changes in the serum, the features are highly predictive of disease.

One model, combining 10 features yielded 98.5% specificity and 90.3% sensitivity on a testing set of 196 non-malignant sera and 103 invasive sera, which dropped to 94.4% specificity (95% CI 83.7-98.6%) and 80.5% sensitivity (95% CI 64.6-90.6%) on a truly blinded validation set of 54 non-malignant and 41 invasive sera.

### CONCLUSION

Serum profiling using this technology and algorithm is reasonably accurate in classifying women with breast abnormalities prior to undergoing biopsy.

### EXAMPLE 2

### Methods

Samples were collected and processed in a manner similar to those described in Example 1, and included 419 Normal Benign sera and 276 Invasive Cancer sera. Spectra were collected in the 200 to 1100 m/z range. From these serum samples, a second randomly selected group was held out as a second independent validation set (i.e., 60 Normal benign and 39 Invasive Cancer spectra. Mass spectrometry was performed on a QSTAR-XL (API 4000, Applied Biosystems/Sciex) equipped with an ABI Turbo-ESI source set at 400 C, a Rheos CPS-LC Pump (2000, Flux Instruments) and a CTC PAL temperature controlled autosampler from LEAP Technologies. ProteomeQuest® software was used to process spectral files from these samples. Approximately 5% of the spectra were excluded based upon concerns such as poor alignment, signal strength and signal to noise ratios.

### Results

A number of models were created which again demonstrated sensitivities and specificities in the range of 80-90% on the blinded validation set. We identified an additional region in the 200 to 500 m/z spectral range that presented m/z features of significant discriminating value, and more particularly in the 200 to 300 m/z range and in the 400 to 400 m/z range. Specifically, m/z peaks of particular discriminating value were found at and around 235.5 and 275.5. These relatively lower m/z feature reflect metabolomic molecules in the blood serum, rather than the blood serum proteins. One, two, or preferably three or more features could be found in the metabolomic range. Together with the m/z features in the broader m/z range, these additional features were very powerful in discriminating between invasive cancer and non-malignant conditions.

TABLE A
(+/−2 m/z values)

| | |
|---|---|
| 537 | 1041 |
| 579 | 763 |
| 1015 | 1093 |
| 543 | 1005 |
| 811 | 1049 |
| 827 | 1069 |
| 545 | 807 |
| 785 | 919 |
| 703 | 521 |
| 737 | 659 |
| 1055 | 813 |
| 739 | 1029 |
| 783 | 1053 |
| 1043 | 1091 |
| 519 | 595 |
| 521 | 731 |
| 741 | 769 |
| 787 | 875 |
| 829 | |
| 879 | |
| 803 | |
| 855 | |
| 909 | |
| 941 | |
| 523 | |
| 833 | |
| 907 | |
| 1049 | |
| 553 | |
| 555 | |
| 619 | |
| 727 | |
| 805 | |
| 853 | |
| 937 | |
| 1051 | |
| 539 | |
| 827 | |
| 997 | |
| 579 | |
| 1031 | |
| 543 | |
| 829 | |
| 761 | |
| 785 | |
| 783 | |

TABLE 1
Normal/Benign Sera

| | Sample | Proportion of |
|---|---|---|
| Status | Number | Samples |
| Normal | 30 | 7.2% |
| Benign, | 231 | 55.3% |
| Non- | | |
| neoplastic | | |
| Benign, | 128 | 30.6% |
| Neoplastic | | |
| Atypical | 29 | 6.9% |
| Hyperplasia | | |
| TOTAL: | 418 | |

TABLE 2
Breast Cancer Sera

| | Sample | Proportion of |
|---|---|---|
| Stage | Number | Samples |
| Stage 0 | 95 | 26.7% |
| DCIS | 57 | 59.8% |
| LCIS | 28 | 30.4% |
| DCIS/LCIS | 9 | 8.7% |
| Other | 1 | 1.1% |
| Stage 1 | 138 | 38.8% |
| Stage 2 | 77 | 21.6% |
| Stage 3 | 30 | 8.4% |
| Stage 4 | 11 | 3.1% |
| Unknown | 5 | 1.4% |
| TOTAL: | 356 | |

1. A model for classifying a biological sample taken from a mammalian subject into one of at least two possible biological states related to breast cancer using a data stream that is obtained by performing a mass spectral analysis of the biological sample, the data stream including magnitude values for a range of mass-to-charge values, comprising:
at least one classifying hypervolume associated with one of the at least two biological states related to breast cancer and disposed within a vector space having n dimensions, each dimension corresponding to a different mass-to-charge value;
wherein n is at least three and at least a first of the dimensions corresponds to a mass-to-charge value in a range of m/z values selected from the m/z ranges consisting of between 200 to 300, 300 to 400, 400 to 500, 500 to 600, 600 to 700, and 700 to 900.

2. The model of claim 1, wherein n is at least 5.

3. The model of claim 1, wherein n is between 5 and 25.

4. The model of claim 1, wherein at least a second of the dimensions corresponds to a mass-to-charge value of between 500 and 1100.

5. The model of claim 1, wherein at least a second of the dimensions corresponds to a mass-to-charge value of between 500 and 900.

6. The model of claim 1, wherein at least a second of the dimensions corresponds to a mass-to-charge value of between 700 and 900.

7. The model of claim 1, the at least one classifying hypervolume being a first classifying hypervolume, further comprising:
a second classifying hypervolume disposed within the vector space;
the first classifying hypervolume being associated with a presence of breast cancer, the second classifying hypervolume being associated with an absence of breast cancer.

8. The model of claim 1, the at least one classifying hypervolume being a first classifying hypervolume, further comprising:
a second classifying hypervolume disposed within the vector space;
the first classifying hypervolume and the second classifying hypervolume being associated with a presence of breast cancer.

9. The model of claim 1, the at least one classifying hypervolume being a first classifying hypervolume, further comprising:
a second classifying hypervolume disposed within the vector space;
the first classifying hypervolume and the second classifying hypervolume being associated with an absence of breast cancer.

10. The model of claim 1, wherein the classifying hypervolume is associated with a presence of breast cancer.

11. The model of claim 10, wherein the classifying hypervolume is associated with a presence of in situ breast cancer.

12. The model of claim 10, wherein the classifying hypervolume is associated with a presence of invasive breast cancer.

13. The model of claim 10, wherein the classifying hypervolume is associated with a likelihood of metastasis of the invasive breast cancer.

14. The model of claim 1, wherein the classifying hypervolume is associated with an absence of breast cancer.

15. The model of claim 14, wherein the classifying hypervolume is associated with a benign breast condition.

16. The model of claim 14, wherein the benign breast condition is selected from the group consisting of hyperplasia, radial scar, calcification, and fibroadenoma.

17. The model of claim 14, wherein the classifying hypervolume is associated with a likelihood of a future occurrence of breast cancer

18. The model of claim 1, wherein the model has at least a 65% accuracy.

19. The model of claim 1, wherein the model has at least a 70% accuracy.

20. The model of claim 1, wherein the model has at least a 80% sensitivity.

21. The model of claim 1, wherein the model has at least a 80% specificity.

22. The model of claim 1, where in the hypervolume is a hypersphere.

23. A method of classifying a biological sample taken from a subject into one of at least two possible biological states related to breast cancer by analyzing a data stream that is obtained by performing a mass spectral analysis of the biological sample, the data stream including magnitude values for a range of mass-to-charge values, comprising:
abstracting the data stream to produce a sample vector that characterizes the data stream in a vector space having n dimensions and containing a diagnostic hypervolume, the vector space having at least a first dimension, a second dimension, and a third dimension, the first dimension corresponding to a mass-to-charge value of between 500 and 600, the second dimension corresponding to a mass-to-charge value of between 700 and 900, the diagnostic hypervolume corresponding to one of the presence or absence of breast cancer; and
determining whether the sample vector rests within the diagnostic hypervolume.

24. The method of claim 23, wherein the hypervolume corresponds to the presence of breast cancer and further comprising:
if the sample vector rests within the diagnostic hypervolume, identifying the biological sample as indicating that the subject has breast cancer.

25. The method of claim 23, wherein the third dimension corresponds to a mass-to-charge value of between 500 and 1100.

26. The method of claim 23, wherein the third dimension corresponds to a mass-to-charge value of between 500 and 900.

27. The method of claim 23, the diagnostic hypervolume is a first diagnostic hypervolume, wherein the vector space contains a second diagnostic hypervolume, the first diagnostic hypervolume and the second diagnostic hypervolume corresponding to the presence of breast cancer.

28. The model of claim 23, the diagnostic hypervolume is a first diagnostic hypervolume corresponding to the presence of breast cancer, wherein the vector space contains a second diagnostic hypervolume, the second diagnostic hypervolume corresponding to an absence of breast cancer.

29. The method of claim 23, wherein the hypervolume is a hypersphere.

30. The method of claim 23, wherein the hypervolume corresponds to the presence of in situ breast cancer.

31. The method of claim 23, wherein the hypervolume corresponds to the presence of invasive breast cancer.

32. The method of claim 24, wherein the hypervolume corresponds to the absence of breast cancer and to the presence of a benign breast condition.

33. The model of claim 32, wherein the benign breast condition is selected from the group consisting of hyperplasia, radial scar, calcification, and fibroadenoma.

34. A model for classifying a biological sample taken from a mammalian subject into one of at least two possible biological states related to breast cancer using a data stream that is obtained by performing an mass spectral analysis of the biological sample, the data stream including magnitude values for a range of mass-to-charge values, comprising:
at least one classifying hypervolume disposed within an vector space having n-dimensions, each dimension corresponding to a different mass-to-charge value,
wherein n is greater than three, at least two of the dimensions correspond to mass-to-charge values in table A.

35. The model of claim 34, wherein at least three of the dimensions correspond to mass-to-charge values in table A.

36. The model of claim 34, wherein n is between 5 and 25.

37. The model of claim 34, the at least one classifying hypervolume being a first classifying hypervolume, further comprising:
a second classifying hypervolume disposed within the vector space,
the first classifying hypervolume being associated with a presence of breast cancer, the second classifying hypervolume being associated with an absence of breast cancer.

38. The model of claim 34, the at least one classifying hypervolume being a first classifying hypervolume, further comprising:
a second classifying hypervolume disposed within the vector space,
the first classifying hypervolume and the second classifying hypervolume being associated with a presence of breast cancer.

39. The model of claim 34, the at least one classifying hypervolume being a first classifying hypervolume, further comprising:
a second classifying hypervolume disposed within the vector space,
the first classifying hypervolume and the second classifying hypervolume being associated with an absence of breast cancer.

40. The model of claim 34, wherein the classifying hypervolume is associated with a presence of breast cancer.

41. The model of claim 40, wherein the classifying hypervolume is associated with a presence of in situ breast cancer.

42. The model of claim 40, wherein the classifying hypervolume is associated with a presence of invasive breast cancer.

43. The model of claim 42, wherein the classifying hypervolume is associated with a likelihood of metastasis of the invasive breast cancer.

44. The model of claim 34, wherein the classifying hypervolume is associated with an absence of breast cancer.

45. The model of claim 44, wherein the classifying hypervolume is associated with a benign breast condition.

46. The model of claim 45, wherein the benign breast condition is selected from the group consisting of hyperplasia, radial scar, calcification, and fibroadenoma.

47. The model of claim 44, wherein the classifying hypervolume is associated with a likelihood of a future occurrence of breast cancer.

48. The model of claim 34, wherein the model has at least a 65% accuracy.

49. The model of claim 34, wherein the model has at least a 70% accuracy.

50. The model of claim 34, wherein the model has at least a 80% sensitivity.

51. The model of claim 34, wherein the model has at least a 80% specificity.

52. A model for classifying a biological sample taken from a mammalian subject using a data stream that is obtained by performing a mass spectral analysis of the biological sample, the data stream including magnitude values for a range of mass-to-charge values, comprising:
at least one classifying hypervolume disposed within a vector space having n dimensions, each dimension corresponding to a different mass-to-charge value,
wherein n is at least three, at least a first of the dimensions corresponds to a mass-to-charge value of between 500 and 600, at least a second of the dimensions corresponds to a mass-to-charge value of between 600 and 700.

53. The model of claim 52, wherein n is at least 5.

54. The model of claim 52, wherein n is between 5 and 25.

55. The model of claim 52, wherein the model has at least a 65% accuracy.

56. The model of claim 52, wherein the model has at least a 70% accuracy.

57. A model for classifying a biological sample taken from a mammalian subject using a data stream that is obtained by performing a mass spectral analysis of the biological sample, comprising:
at least two classifying hypervolumes disposed within a vector space having at least three dimensions, one of the at least two classifying hypervolumes being associated with a presence of a disease, another of the at least two classifying hypervolumes being associated with an absence of the disease,
the model having at least a 65% accuracy.

58. The model of claim 57, wherein the vector space has at least 5 dimensions.

59. The model of claim 57, wherein the disease is breast cancer.

60. The model of claim 57, wherein the data stream includes magnitude values for a range of mass-to-charge values, a first of the at least three dimensions corresponds to a mass-to-charge value of between 500 and 600, and a second of the at least three dimensions corresponds to a mass-to-charge value of between 600 and 700.

61. The model of claim 57, wherein the data stream includes magnitude values for a range of mass-to-charge values, at least two of the at least three dimensions correspond to mass-to-charge values in table 1.

62. The model of claim 1, wherein the first of the dimensions corresponds to a mass-to-charge value of between 520 and 590.

63. The model of claim 1, wherein the first of the dimensions corresponds to a mass-to-charge value of about 537.

64. The model of claim 1, wherein the first of the dimensions corresponds to a mass-to-charge value of about 579.

65. The model of claim 1, wherein the first of the dimensions corresponds to a mass-to-charge value of between 535 and 540.

66. The model of claim 1, wherein the first of the dimensions corresponds to a mass-to-charge value of between 575 and 580.

67. The model of claim 1, wherein the second of the dimensions corresponds to a mass-to-charge value of about 827.

68. The model of claim 1, wherein the second of the dimensions corresponds to a mass-to-charge value of between 820 and 830.

69. A model for classifying a biological sample taken from a mammalian subject into one of at least two possible biological states using a data stream that is obtained by performing a mass spectral analysis of the biological sample, the data stream including magnitude values for a range of mass-to-charge values, comprising:
at least one classifying hypervolume associated with the presence of ductal carcinoma in situ and disposed within a vector space having n dimensions, each dimension corresponding to a different mass-to-charge value.

70. The model of claim 69, wherein n is at least three, at least a first of the dimensions corresponds to a mass-to-charge value of between 900 and 905, and at least a second of the dimensions corresponds to a mass-to-charge value of between 610 and 620.

71. The model of claim 69, the at least one classifying hypervolume being a first classifying hypervolume, further comprising:
a second classifying hypervolume associated with the presence of lobular carcinoma in situ and disposed within the vector space.

72. A model for classifying a biological sample taken from a mammalian subject into one of at least two possible biological states using a data stream that is obtained by performing a mass spectral analysis of the biological sample, the data stream including magnitude values for a range of mass-to-charge values, comprising:
at least one classifying hypervolume associated with the presence of lobular carcinoma in situ and disposed within a vector space having n dimensions, each dimension corresponding to a different mass-to-charge value.

73. The model of claim 72, wherein n is at least three, at least a first of the dimensions corresponds to a mass-to-charge value of between 1050 and 1060, and at least a second of the dimensions corresponds to a mass-to-charge value of between 610 and 620.

74. A model for classifying a biological sample taken from a mammalian subject into one of at least two possible biological states associated with breast pathology using a data stream that is obtained by performing a mass spectral analysis of the biological sample, the data stream including magnitude values for a range of mass-to-charge values, comprising:
at least one classifying hypervolume disposed within a vector space having n dimensions, each dimension corresponding to a different mass-to-charge value.
